(19) United States
(12) Patent Application Publication
(10) Pub. No.: US 2026/0110014 A1
(43) Pub. Date: Apr. 23, 2026

(54) METHOD FOR CLINICAL URINARY TRACT INFECTION AND BACTEREMIA DETECTION

(71) Applicant: The University of South Alabama, Mobile, AL (US)

(72) Inventors: Allyson Shea, Mobile, AL (US); Rachel Fleck, Mobile, AL (US)

(73) Assignee: The University of South Alabama, Mobile, AL (US)

(21) Appl. No.: 19/312,924

(22) Filed: Aug. 28, 2025

Related U.S. Application Data
(60) Provisional application No. 63/710,868, filed on Oct. 23, 2024.
(60) Provisional application No. 63/794,905, filed on Apr. 25, 2025.

(51) Int. Cl. C12Q 1/10; C12Q 1/08; C12Q 1/14; G01N 1/38

(52) U.S. Cl. CPC C12Q 1/10; C12Q 1/08; C12Q 1/14; G01N 1/38; G01N 2800/348

(57) ABSTRACT

A diagnostic kit and method for detecting urinary tract infections (UTIs) and bacteremia using a drinking water test in low-resource settings is provided. The kit includes a first bag containing a first culture medium capable of detecting Gram-negative and coliform bacteria through colorimetric and fluorescence indicators, and a second bag containing a second culture medium capable of detecting Gram-positive bacteria through colorimetric indicators. A method of using the kit involves inoculating a sample into each bag, incubating the bags, and observing for color changes and fluorescence to indicate bacterial presence. The kit enables rapid, cost-effective detection of bacterial pathogens in urine and blood samples without specialized laboratory equipment, facilitating early diagnosis and treatment in resource-limited environments.

### CROSS REFERENCES

This application claims the benefit of U.S. Provisional Application No. 63/794,905, filed on Apr. 25, 2025, and of U.S. Provisional Application No. 63/710,868, filed Oct. 23, 2024, which applications are herein incorporated entirely by reference.

### FIELD OF INVENTION

The present disclosure relates to diagnostic test kits for detecting bacterial pathogens, and more particularly to a modified water quality test kit for diagnosing urinary tract infections and bloodstream infections in low-resource settings.

### BACKGROUND

Urinary tract infections (UTIs) comprise the second most common infectious disease worldwide and a major cause of hospital-acquired sepsis, itself a leading cause of death. Early diagnosis and prompt treatment of a UTI is crucial for preventing deadly or irreversibly damaging complications. However, diagnosing the specific cause of a UTI without access to laboratory facilities presents significant challenges for healthcare providers, particularly in resource-limited settings or remote areas. The ability to accurately identify the pathogen responsible for a UTI is crucial for determining appropriate treatment and preventing complications. Accurate pathogen identification allows healthcare providers to select the most effective antibiotic therapy, reducing the risk of treatment failure and the development of antibiotic resistance.

Different bacterial species causing UTIs may have varying susceptibility profiles to commonly used antibiotics. For example, Escherichia coli, the most common cause of UTIs, may have different resistance patterns compared to other pathogens like Klebsiella or Proteus species. Precise identification can also help distinguish between uncomplicated and complicated UTIs, which may require different treatment approaches or durations. For instance, the presence of Gram-positive Staphylococcus species is a common feature of complicated UTIs, though they are relatively uncommon in uncomplicated UTI. Furthermore, accurate pathogen identification is essential for detecting and managing recurrent or persistent infections, as well as identifying potential underlying urological abnormalities that may predispose patients to UTIs. In resource-limited settings, where broad-spectrum antibiotics may be overused due to diagnostic uncertainty, the ability to accurately identify pathogens could significantly improve antibiotic stewardship efforts and patient outcomes.

Despite UTI prevalence in areas with poor healthcare coverage, traditional methods of diagnosing UTIs often rely on laboratory culture and sensitivity testing, which requires specialized equipment and trained personnel. These tests typically take 24 to 48 hours to yield results, during which time patients may experience discomfort and the infection may progress. In settings where laboratory access is limited or unavailable, healthcare providers must often rely on clinical symptoms and basic urinalysis to make treatment decisions, which can lead to misdiagnosis or inappropriate antibiotic use. The lack of rapid, accurate diagnostic tools for UTIs in non-laboratory settings can contribute to several issues, including delayed treatment, overuse of broad-spectrum antibiotics, and the potential development of antibiotic resistance. Additionally, the inability to differentiate between various pathogens can make it difficult to track epidemiological trends and implement targeted public health interventions. Interestingly, there is overlap between bacteria tested for in drinking water test kits and bacteria that commonly cause bacterial UTI. Biological media test kits configured to test drinking water are commonly available and inexpensive, meaning there is potential that drinking water tests kits could be repurposed to test for UTIs in areas with limited or unavailable laboratory access.

Accordingly, there is a need in the art for a system and method for repurposing drinking water test kits to be innovative, portable, and easy-to-use diagnostic solutions for determining bacterial infections.

### SUMMARY

A system and method for preparing blood and urine samples and testing them with a biological media test kit to diagnose urinary tract infections or bacteremia is provided. In one aspect, the present invention is a way of diversifying the function of commonly available water testing kits, allowing communities of limited means to flexibly apply their resources to clinical diagnostics. In another aspect, the present invention is a method of preparing urine samples to assess the presence or absence of bacterial infection in the urinary tract. In yet another aspect, the present invention is a method of preparing multifunctional blood samples to assess bacterial infection constituting bacteremia. Generally, the present invention is a method for diagnosing bacterial UTI or bacteremia without the need for controlled laboratory conditions by repurposing a biological media test kit designed to test drinking water for bacterial contamination.

The system generally comprises a biological media test kit and a mannitol salt broth (MSB) kit, wherein the biological media test kit is configured to test for Gram-negative bacteria in drinking water and the MSB kit is configured to supplement the biological media test kit by testing for Gram-positive bacteria. Gram-positive bacteria detectable by the MSB kit primarily comprise Staphylococcus species in general, differentiating Staphylococcus aureus in particular. The method begins by collecting a urine sample, a blood sample, or both from a subject. The sample is used to generate a dilute preparation of biological material in water or media. The latter is preferably accomplished by taking a small volume of said sample and introducing it into it into sterile water, into which prepared solid or powdered media-obtained from one of the biological media test kits-is mixed. For the most complete analysis, the sample is tested with media from a biological media test kit and an MSB kit in separate aliquots. The inoculated media is subsequently incubated in ambient conditions for a period of time appropriate for assessing the growth of bacteria. After the appropriate incubation, the inoculated media is assessed for bacterial growth by one or more indicia of bacterial presence. Said indicia are determined by the composition of the media, the characteristics of the bacteria the user is attempting to detect, and the conditions of the incubation.

The foregoing summary has outlined some features of systems and methods used to determine when microbiological transfer has occurred so that those skilled in the pertinent art may better understand the detailed description that follows. Additional features that form the subject of the claims will be described hereinafter. Those skilled in the pertinent art should appreciate that they can readily utilize these features for preparing and testing other biological samples such as plasma, serum, or mucosal swabs. Those skilled in the pertinent art should also realize that such equivalent designs or modifications do not depart from the scope of the method of the present disclosure.

### BRIEF DESCRIPTION OF FIGURES

These and other features, aspects, and advantages of the present disclosure will become better understood with regard to the following description, appended claims, and accompanying drawings where:

FIG. 1 illustrates a diagram outlining the basic aspects of utilizing a biological media test kit to test biological samples consistent with the principles of the present disclosure.

FIG. 2 illustrates a diagram describing the method of preparing urine samples for testing with a biological media test kit consistent with the principles of the present disclosure.

FIG. 3 illustrates a diagram describing the method of preparing blood samples for testing with a biological media test kit consistent with the principles of the present disclosure.

FIG. 4 illustrates a diagram describing the method of using an Aquagenx® CBT EC+TC P/A kit consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

In accordance with long standing patent law convention, the words “a” and “an” when used in this application, including the claims, denote “one or more. ”

The term “comprises” and grammatical equivalents thereof are used herein to mean that other components, steps, etc. are optionally present. For example, a buffer solution “comprising” components A, B, and C can contain only components A, B, and C, or can contain not only components A, B, and C, but also one or more other components.

The term “consisting of” and grammatical equivalents thereof are used herein to mean that other components, steps, etc. are not optionally present. For example, a buffer solution “consisting of” components A, B, and C can only contain components A, B, and C.

The terms “about” and “approximately,” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

β-galactosidase: An enzyme capable of catalyzing the hydrolysis of β-D-galactose residues from the terminal non-reducing ends of β-D-galactosides like lactose. The ability to produce β-galactosidase is one of the distinguishing features of coliform bacteria.

β-glucuronidase: An enzyme capable of catalyzing the hydrolysis of β-D-glucuronic acid residues from the non-reducing end of glycosaminoglycans. The presence of β-glucuronidase is a distinguishing feature of E. coli, while rarely expressed by unrelated coliforms. As such, a skilled artisan would know that the presence of β-glucuronidase is an excellent indicator of the presence of E. coli without microscopic confirmation.

Bacteremia: the condition of having bacteria in the bloodstream. Notably, any number of bacteria in the bloodstream, however small, constitutes an abnormality and an instance of infection. While in many instances the immune system rapidly removes the infecting bacteria, failure to rapidly clear the infection can lead to sepsis, a life-threatening condition.

Biological Media Test Kit (BMT kit): A prefabricated assembly sold, given, or transferred as a unit comprising some or all of the reagents and equipment necessary to detect the presence of one or more microorganisms or types or microorganism by means of culture. BMT kits may comprise one or more of the following: powdered media, solid media, semisolid media, liquid media, media supplements, vessels for culturing, chemical indicators, biological indicators, antibiotics, sampling means such as needles or urine cups, inoculation loops, cleaning or antiseptic means, washes, and other components facilitating kit use.

Coliform: A bacterium belonging to a group of several genera (e.g., Citrobacter, Enterobacter, Hafnia, Serratia, Klebsiella, and Escherichia) of Gram-negative rod-shaped bacteria that have the ability to ferment lactose with a resultant production of acid and gas. Most coliform bacteria are generally considered nonpathogenic to humans. However, some coliform bacteria (e.g., Escherichia coli) include strains that are highly pathogenic. Coliforms are found in the fecal matter of mammals and are commonly used as an indicator of fecal contamination of food and/or water. Some strains of coliform bacteria are common causative agents of urinary tract infection, particularly Escherichia coli and Klebsiella pneumoniae.

Culture: The method of multiplying microbial organisms by letting them reproduce in predetermined culture media under conditions conducive for their growth. More particularly it is the method of providing a suitable culture medium and conditions to facilitate at least one cell division of a microorganism. Culture media are solid, semisolid or liquid media containing all of the nutrients and necessary physical growth parameters necessary for microbial growth. Culture may also refer to preparations of culture media in which microbes have been cultivated already.

Enrichment: The culture method of selectively enhancing the growth of a specific microorganism by providing medium and conditions with specific and known attributes that favors the growth of that particular microorganism. The enrichment culture's environment will positively influence the growth of a selected microorganism and/or negatively influence the growth of other microorganisms.

Enzyme: A biological catalyst comprising one or more polypeptides. Methods of manufacturing and utilizing enzymes are known to the skilled artisan and are necessary for the success of modifying the method disclosed herein.

Fluorescence: The ability to emit light of a particular wavelength (emission wavelength) when exposed to light of another wavelength (excitation wavelength). A common technique in biological tests, kits, or assays that assess the presence of two or more factors is to couple one factor to a colorimetric dye and another factor to a fluorescent dye; accordingly, one can assess the presence of one factor via color change and the other by fluorescence. Fluorescent dyes and proteins with distinct excitation and emission properties are familiar to the skilled artisan; for example, functional GFPs, CFPs and YFPs comprise distinct excitation and emission properties (see e.g., Tsien, Annu. Rev. Biochem., 67:509-544, 1998.)

Gram-positive: Referring to bacteria that retain the crystal violet stain used in the Gram staining method due to the thick peptidoglycan layer in their cell walls and lack of an outer membrane. Gram-positive bacteria appear purple under microscopic examination after Gram staining. Common Gram-positive bacteria include Staphylococcus species (including S. aureus and S. epidermidis), Streptococcus species, and Enterococcus species. Some Gram-positive bacteria, particularly Staphylococcus aureus, can cause urinary tract infections, especially in complicated cases, and are also significant causative agents of bacteremia and sepsis.

Gram-negative: Referring to bacteria that do not retain the crystal violet stain used in the Gram staining method due to their thin peptidoglycan layer and presence of an outer membrane containing lipopolysaccharides. Gram-negative bacteria appear pink or red under microscopic examination after Gram staining. Common Gram-negative bacteria include Escherichia coli, Klebsiella species, Proteus species, and Pseudomonas aeruginosa. These bacteria are frequent causative agents of urinary tract infections and can also cause bacteremia and sepsis.

Mannitol salt broth: A selective and differential liquid growth medium used for the isolation and identification of Staphylococcus species, particularly Staphylococcus aureus. The medium typically contains a high concentration of sodium chloride (7.5-10%), which inhibits the growth of most bacteria except for halotolerant organisms like Staphylococcus. It also contains mannitol as a carbohydrate source and phenol red as a pH indicator. S. aureus can ferment mannitol, producing acid that changes the color of the medium from red to yellow, while most other Staphylococcus species cannot ferment mannitol and thus do not change the medium's color. The skilled artisan will recognize that solid and semisolid variants of this medium may be employed without departing from the subject matter described herein.

Protein or Polypeptide: A polymer of amino acid residues, including amino acid polymers in which one or more amino acid residue is an artificial chemical mimetic of a corresponding naturally occurring amino acid, as well as to naturally occurring amino acid polymers and non-naturally occurring amino acid polymers. Multiple polymers of amino acids binding to each other are a protein complex. Protein and polypeptide may be used interchangeably throughout this application and mean at least two covalently attached amino acids, which includes proteins, polypeptides, oligopeptides and peptides.

Sepsis: A life-threatening organ dysfunction due to a dysregulated host response to infection. Pathogenesis of the sepsis syndrome relies critically on the activation of innate immunity by a large family of pattern recognition receptors (PRRs) in response to microbial pathogens, including especially Gram-negative bacilli such as E. coli and P. aeruginosa.

Urinary tract infection (UTI): An infection in any part of the urinary system. The urinary system of a human comprises the kidneys, ureters, bladder, and urethra. Most UTI's involve the lower urinary tract, including the bladder and the urethra, but infections can spread to the ureters, kidneys, or prostate gland. Urinary tract infection is frequently painful and annoying; furthermore, left untreated a UTI can progress to cause serious health problems such as sepsis, nephritis, or infertility. While the classic symptoms of a UTI include a burning sensation during urination, UTI's can occur without the patient experiencing this symptom.

Urosepsis: Sepsis originating from a UTI, frequently by contamination of the blood with live bacteria or dead bacterial components.

### Methods for Utilizing an Aquagenx® Kit to Test for E. coli, S. aureus, or Other Staphylococcus or Coliform-Mediated UTI

The present invention addresses the impediments common in field UTI diagnosis by repurposing a WHO-certified drinking water test kit for diagnostic purposes. Drinking water test kits/water quality field test kits were initially developed to serve as a fast, flexible, low-cost means of identifying potentially hazardous bacteria in drinking water supplies. However, there is considerable overlap between bacteria hazardous in drinking water and the common causes of bacterial UTI and bacteremia. As such, the drinking water test kits may be applied as a diagnostic tool for clinical samples without necessitating laboratory equipment or controlled environmental conditions which may or may not be accessible. In this way, the present invention provides medical personnel with a valuable tool for shaping UTI diagnosis in underserved communities.

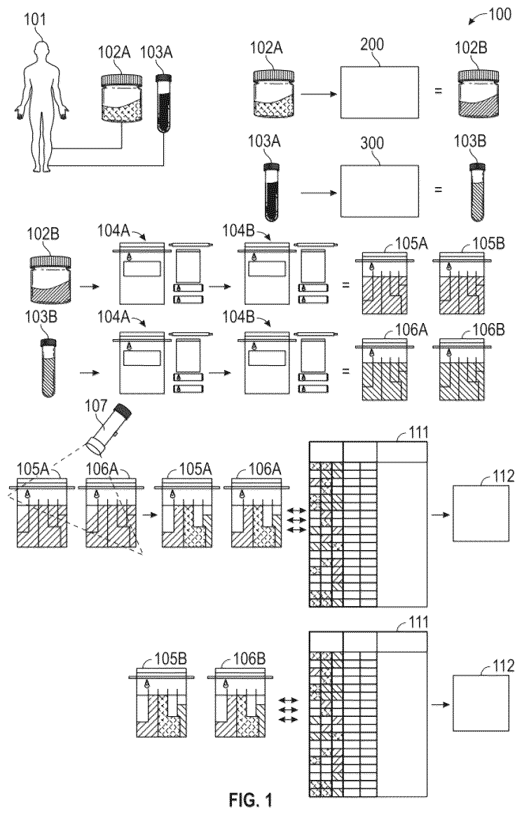

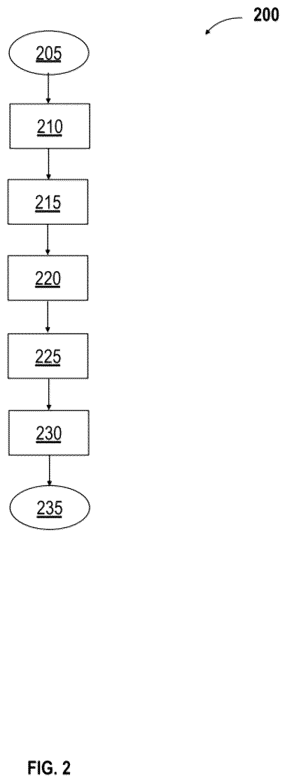

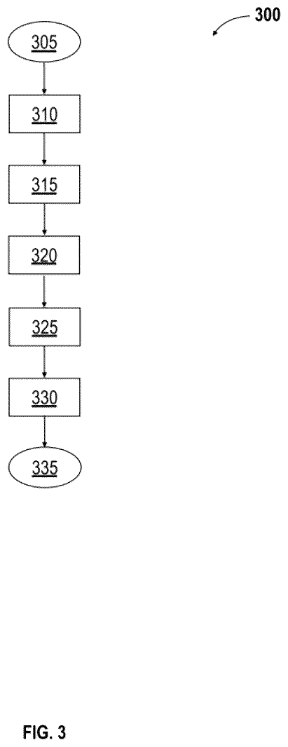

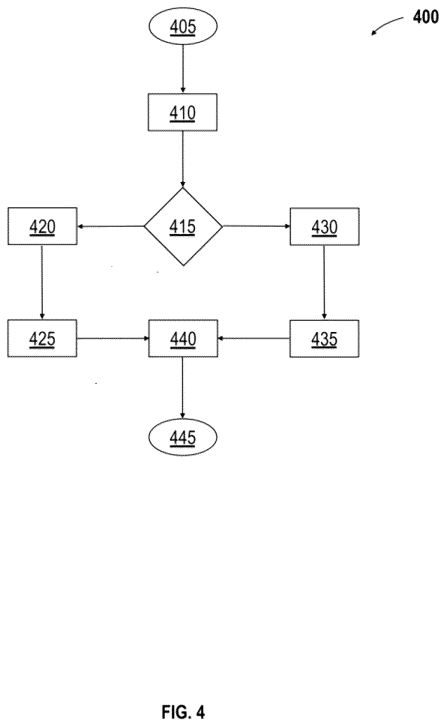

FIGS. 1-4 illustrate certain, preferred embodiments for the method of utilizing a biological media test kit to test for the presence of bacteria in blood or urine. FIG. 1 illustrates the overall system and method, with three sections themselves comprising distinguishable methods detailed later herein. FIG. 2 details the method of preparing a biological sample comprising urine such that it may be used by the biological media test kit. FIG. 3 details the method of preparing a biological sample comprising blood such that it may be used by the biological media test kit. FIG. 4 details the method of utilizing a biological media test kit to test for the presence of bacteria in clinical samples. In a preferred embodiment, the biological media test kit so utilized is a drinking water test kit. In another preferred embodiment, the drinking water test kit so utilized is the Aquagenx® CBT EC+TC P/A kit. In yet another preferred embodiment, the drinking water test kit so utilized is the Aquagenx® CBT EC+TC MPN Kit. In yet another preferred embodiment, the drinking water test kit so used is the CBT AMR EC MPN Kit. In yet another preferred embodiment, the CBT AMR P/A Kit. In still another preferred embodiment, the drinking water test kit is supplemented with a bag comprising mannitol salt broth (MSB) media.

The standard formulation for mannitol salt broth is well-known to the skilled artisan. In a preferred embodiment, the present methods use the standard mannitol salt broth formulation. In another preferred embodiment, however, the present methods use a novel modified formulation to enhance bacterial detection. For instance, the standard 10 g/L of proteose peptone is preferably replaced with 10 g/L tryptone peptone. Furthermore, 1 g/L yeast extract may be used to replace the standard 1 g/L beef extract.

### Overall Diagnostic Method

FIG. 1 illustrates the system 100 for determining bacterial infections and details the diagnostic methods utilizing a repurposed biological media testing kit 104A (BMT kit) to assess the presence of bacteria in biological samples. In a preferred embodiment, the BMT kit 104A so utilized is the Aquagenx® CBT EC+TC P/A kit, which is designed to test drinking water for the presence E. coli and other coliforms. In another preferred embodiment, the BMT kit 104A is supplemented with a MSB kit 104B. The method begins with the subject 101. In a preferred embodiment, the subject 101 is a human who is suspected of having a UTI or bacteremia. In a majority of preferred embodiments, the subject 101 is assisted by one or more medical personnel with the execution of the method described herein. In another embodiment, the subject 101 completes the method unassisted. In yet another preferred embodiment, the subject 101 is given reason to suspect that they might have a UTI due to experiencing symptoms frequently associated with UTIs or bacteremia, e.g. fever, chills, fatigue, discomfort or difficulty urinating, cloudy or discolored urine, or pain in the lower abdomen. In still another preferred embodiment, the subject 101 is given a diagnosis of probable UTI by one or more medical personnel prior to beginning the method.

The subject 101 preferably contributes a urine sample 102A or a blood sample 103A for preparation. In a preferred embodiment, collection of the blood sample 103A is conducted by trained medical personnel using aseptic techniques to minimize the risk of injury or contamination of the sample. In another preferred embodiment, collection of the blood sample 103A is only conducted when one or more medical personnel has reason to suspect that the subject 101 has bacteremia or is at risk of urosepsis. In yet another preferred embodiment, collection of the blood sample 103A occurs at the same time as the collection of the urine sample 102A, but one or more of the blood samples 103A and the urine samples 102A are preferably maintained at temperatures appropriate to slow the growth of, but not kill, bacteria. These samples may be used for diagnostics at a later period or for another purpose. For instance, a study or survey attempting to determine incidence of bacterial UTI or bacteremia during a particular time period might utilize previously collected samples 102A or 103A to make projections about hazards that coliforms or Staphylococcus species pose to subjects 101. In a preferred embodiment, all samples 102A, 103A are stored in sterile vessels until prepared.

Subsequent to collection, the samples 102A, 103A are prepared for testing, which preferably involves diluting in sterile water. The source and characteristics of the sterile water may vary. In a preferred embodiment, the sterile water is laboratory grade distilled and deionized water. In another preferred embodiment, the sterile water is sealed, bottled water the user has reasonable cause to believe is sterile. In yet another highly preferred embodiment, the sterile water is sterilized by means of boiling or autoclaving. One benefit of boiling or autoclaving said water is that even water of dubious origin and questionable purity may be rendered safe for use in the kit, reducing the odds of generating a false positive. Furthermore, even low-resource communities often have the means of boiling water; as such, the kit may be utilized in the field without access to laboratory grade water.

The urine sample 102A is preferably prepared using method 200, detailed in FIG. 2. Method 200 yields a urine preparation 102B, which is suitable for testing with the BMT kit 104A and the MSB kit 104B. The blood sample 103A is preferably prepared using method 300, which is detailed in FIG. 3. Method 300 yields a blood preparation 103B, which is suitable for testing with the BMT kit 104A and the MSB kit 104B. In a preferred embodiment, method 200 and method 300 occur on-site immediately following collection of the urine samples 102A or blood samples 103A. In another preferred embodiment, one or more medical personnel perform method 200 or method 300 on behalf of the subject 101 to reduce the risk of mishandling or contamination. In still another preferred embodiment, the samples 102, 103 are removed to another location for testing with the BMT kit 104, either prior to or following method 200 or method 300.

The urine preparation 102B or blood preparation 103B are preferably tested using the BMT kit 104A, wherein the method for doing such is described in more detail below via method 400 and in FIG. 4. In a preferred embodiment, the urine preparation 102B and blood preparation 103B are also tested with the MSB kit 104B. Completion of method 400 on the urine preparation 102B or the blood preparation 103B yields a urine culture 105 or a blood culture 106, respectively. The type of urine culture or blood culture produced is dependent on the kit 104 used to test the blood and urine preparations. Urine and blood preparations tested using the BMT kit 104A produce urine coliform cultures 105A and blood coliform cultures 106A, respectively. Urine and blood preparations tested with the MSB kit 104B produce urine Staphylococcus cultures 105B and blood Staphylococcus cultures 106B, respectively.

Cultures 105, 106 may then be analyzed to assess indicia of bacterial presence. Indicia of bacterial presence are determined by a number of factors, including but not limited to the characteristics of the cultured bacteria, the composition of the BMT kit 104A and the MSB kit 104B, and the conditions of incubation executed in method 400. In a preferred embodiment, indicia of coliform or Staphylococcus bacterial presence in the cultures 105, 106 comprise one or more of luminescence, turbidity, color change, clumping, and odor. In another preferred embodiment, the indicia of infection comprise acquisition of fluorescence, change of color to green or black, and increased turbidity. In yet another preferred embodiment, each indicium of bacterial presence is indicative of a different bacterium or clade of bacteria, such that different combinations of indicia signify different bacteria or combinations of bacteria.

To assess fluorescence, a UV source 107 briefly illuminates the cultures 105, 106. It is recommended that fluorescence be assessed specifically for urine and blood coliform cultures 105A, 106A. In a preferred embodiment, fluorescence is assessed in an environment with low ambient lighting to prevent a false negative determination. In another preferred embodiment, fluorescence is assessed with a fluorometer. In yet another preferred embodiment, the UV source 107 comprises a UV flashlight. In still another preferred embodiment, the UV source 107 comprises a UV transilluminator.

Generally, color change and turbidity are suitable for visual assessment in ambient lighting, either by examining the color of the media or the presence of sediment. For instance, in embodiments wherein the BMT test 104A is the Aquagenx® CBT EC+TC P/A kit, a blue or green color in the presence of fluorescence indicates the presence of β-glucuronidase and therefore E. coli. In said embodiments, fluorescence without a color change is indicative of non-Escherichia coliform bacteria. Furthermore, turbidity without either fluorescence or a color change indicates the presence of non-coliform Gram-negative bacteria. In other preferred embodiments, the urine and blood Staphylococcus cultures 105B, 106B are assessed for color change and turbidity. Standard mannitol salt broth is phenol red prior to inoculation and undergoes a color change to yellow when mannitol is fermented, generally by S. aureus. Turbidity or sediment without a color change indicates the presence of a halotolerant bacterial infection, most probably a non-S. aureus Staphylococcus species. In a preferred embodiment, culture color is assessed with a colorimetric device.

Upon inspection of the cultures 105, 106, comparisons may be made with a diagnostic key 111. In a preferred embodiment, the diagnostic key 111 comprises images of potential culture colors in the kit or kits 104A-B so that a user may interpret whether the test is positive or negative. In another preferred embodiment, the diagnostic key 111 further comprises descriptions of each color in each kit 104 so that the user may more easily make a determination of presence or absence of bacteria. In yet another preferred embodiment, the diagnostic key 111 further comprises images of media in which a determination cannot be made due to factors like contamination or expiration of reagents. In still another preferred embodiment, the diagnostic key 111 further comprises images and text useful for the interpretation of turbidity or fluorescence results. After comparison with the diagnostic key 111, the user uses the results of the analysis to generate a diagnosis 112. In a preferred embodiment, said analysis and diagnosis are conducted and generated by trained medical personnel. The generation of the diagnosis ends the overall method.

The data acquired by the diagnosis is highly useful for shaping ongoing care for the subject 101. Antibiotic resistance among bacteria is an ongoing concern, particularly in environments where access to supportive/palliative care or multiple antibiotic classes may be limited. Many antibiotics in the β-lactam family show reduced efficacy against Gram-negative bacteria like the coliform genera, so establishing that a UTI is caused by E. coli or another coliform reduces the time necessary to establish a successful treatment regimen while reducing excretion of antibiotics into the environment. Similarly, the presence of S. aureus or related species in urine cultures may indicate that the infection is undergoing complications. The medical professional may then prioritize transporting the patient to more specialized care or supplementing their treatment plan with alternate antibiotics and supportive care.

### Method of Processing Urine Samples for Testing

FIG. 2 illustrates a diagram detailing certain, preferred method steps for the method 200 of processing the urine samples 102A into urine preparations 102B. Step 205 indicates the beginning of the method. During step 210, a urine sample 102A is collected from the subject 101. In a preferred embodiment, the urine is collected in a sterile cup to reduce the risk of contamination and therefore false positives. In another preferred embodiment, the urine sample 102A comprises at least 5 mL of urine to facilitate serial dilutions. In step 215, sterile water is acquired for the purposes of sample preparation. During step 220, the sterile water is allocated in the volumes appropriate for the BMT kit 104A to which it will be applied. In a preferred embodiment, the BMT kit 104 is the Aquagenx® CBT EC+TC P/A kit and therefore 100 mL of water is allocated for its use. In another preferred embodiment, concurrent culture testing is prepared by allocating liquid media comprising compositions distinct from the media contained in the BMT kit 104. In yet another preferred embodiment, the liquid media comprises 100 mL of mannitol salt broth, wherein the MSB is part of a MSB kit 104B.

During step 225, the allocations of sterile water and optional liquid media are transferred into separate sterile, clear plastic bags. In a preferred embodiment, the sterile, clear plastic bags are Whirl-Pak® thio bags sourced from the BMT kit 104A or the MSB kit 104B. In another preferred embodiment, the Whirl-Pak® thio bags are sourced from another supplier, such as Whirl-Pak Filtration Group itself. In yet another preferred embodiment, another sterile, clear, sealable bag of dimensions suitable for holding the urine preparation 102B is used. In step 230, the urine sample 102A is used to inoculate each allocation of sterile water or liquid media in order to generate the urine preparation 102B. In a preferred embodiment, a 10 μL inoculation loop is used to transfer 10 μL of the urine sample 102A into the sterile water or liquid media. In another preferred embodiment, the inoculation loop comprises metal and may therefore be cleaned for reuse by heating, dipping in solutions substantially comprising alcohols, or immersing in flame. In yet another preferred embodiment, the inoculation loop is disposable and discarded after inoculating the sterile water or MSB. In still another preferred embodiment, the sterile water is inoculated by transferring 10 μL of urine via micropipette. If the BMT kit 104A is such that serial dilutions must be performed, such as in the Aquagenx® CBT EC+TC MPN kit, the urine is preferably serially diluted using sterile water prior to inoculating the volume of sterile water, as described by the protocols disclosed by the relevant kit. Step 235 marks the end of the method.

### Method of Processing Blood Samples for Testing

FIG. 3 illustrates a diagram that details certain, preferred method steps of the method 300 for processing blood samples 103A into blood preparations 103B. Step 305 indicates the beginning of the method. During step 310, the blood sample 103A is acquired from the subject 101. In a preferred embodiment, the blood sample 103A is removed using a sterile needle and stored in a sterile container. In another preferred embodiment, the blood sample 103A is removed by a trained medical professional. In still another preferred embodiment, the blood sample 103A is at least 5 mL by volume. In step 315, sterile water is acquired for the purposes of sample preparation. During step 320, the sterile water is allocated in the volumes appropriate for the BMT kit 104A to which it will be applied. In a preferred embodiment, the BMT kit 104 is the Aquagenx® CBT EC+TC P/A kit and, therefore, 100 mL of water is allocated for its use. In another preferred embodiment, concurrent culture testing is prepared by allocating liquid media comprising compositions distinct from the solid media contained in the BMT kit 104A. In yet another preferred embodiment, the liquid media comprises 100 mL of MSB from an MSB kit 104B.

During step 325, the allocations of sterile water and optional liquid media are transferred into separate sterile, clear plastic bags. In a preferred embodiment, the sterile, clear plastic bags are Whirl-Pak® thio bags sourced from the BMT kit 104A or the MSB kit 104B. In another preferred embodiment, the Whirl-Pak® thio bags are sourced from another supplier, such as Whirl-Pak Filtration Group itself. In yet another preferred embodiment, another sterile, clear, sealable bag of dimensions suitable for holding the blood preparation 103B is used. In step 330, the blood sample 103A is used to inoculate each allocation of sterile water or liquid media in order to generate blood preparation 103B. In a preferred embodiment, a transfer pipette allocates up to 5 mL of the blood sample 103A to the sterile water or liquid media. In another preferred embodiment, a pipette aid transfers up to 5 mL of the blood sample 103A to the sterile water or liquid media. In still another preferred embodiment, the sterile water or liquid media is inoculated by transferring up to 5 mL of the blood sample 103A via micropipette. If the BMT kit 104A is such that serial dilutions must be performed, such as in the Aquagenx® CBT EC+TC MPN kit, the blood sample 103A is preferably serially diluted using sterile water prior to inoculating the volume of sterile water, as described by the protocols disclosed by the relevant kit. Step 335 marks the end of the method.

### Method of Testing Blood and Urine Preparations for Bacteria

FIG. 4 illustrates a diagram that details certain, preferred method steps of method 400 for testing the urine preparations 102B or the blood preparations 103B for the presence of bacteria. Step 405 indicates the beginning of the method. In step 410, solid or powdered media is added to those urine preparations 102B or blood preparations 103B which do not already comprise media. In a preferred embodiment, the solid or powdered media is taken exclusively from BMT kit 104A. In another preferred embodiment, the solid or powdered media taken from BMT kit 104A is supplemented with reagents enhancing expression of bacterial growth or differentiation factors. For instance, a kit meant to visualize β-galactosidase-producing coliforms might supplement the media with lactose to ensure robust expression of the differential lac operon. In yet another preferred embodiment, the solid or powdered media is taken from the MSB kit 104B.

In branching step 415, it is determined whether the preparations are urine preparations 102B or blood preparations 103B. Urine preparations 102B proceed to step 420, while blood preparations 103B proceed to step 430. In step 420, the urine preparations 102B are shaken to evenly distribute and dissolve the media in the urine preparation, or to distribute the inoculated urine into the liquid media. In a preferred embodiment, the shaking is performed by hand. In another preferred embodiment, the shaking is performed with the use of a vortex. During step 425, the urine preparations with media are incubated in conditions and for a duration appropriate for the culture of bacteria the user wishes to detect. For instance, if the BMT kit 104A is the Aquagenx® CBT EC+TC P/A kit, which is designed to detect and differentiate E. coli and other coliforms, the urine preparations with media would be incubated at ambient temperatures of approximately 25° Celsius for 20-48 hours. Alternatively, the urine preparations in media from the MSB kit 104B would be incubated in conditions known to the skilled artisan, preferably 18-48 hours. In another preferred embodiment, the incubation takes place at ambient temperatures of approximately 37° Celsius. In a preferred embodiment, incubations of the same urine sample in differing media occur concurrently. For instance, the same urine sample 102A could be used to generate two urine preparations 102B, one in sterile water combined with media from the Aquagenx® CBT EC+TC P/A kit and one comprising MSB from an MSB kit 104B. The 20-48-hour incubation specified by the Aquagenx® kit could then take place during the first 20-48 hours of the incubation necessary for culture in MSB to enable simultaneous readings.

In step 430, the blood preparations 103B are gently inverted to mix and distribute the blood sample within the water and media. In a preferred embodiment, the inversion is performed by hand. In another preferred embodiment, the inversion is performed by a mechanical inverter. During step 435, the blood preparations with media are incubated in conditions and for a duration appropriate for the culture of bacteria the user wishes to detect. For instance, if the BMT kit 104 is the Aquagenx® CBT EC+TC P/A kit, which is designed to detect and differentiate E. coli and other coliforms, the blood preparations with media would be incubated at ambient temperatures of approximately 25° Celsius for 20-48 hours. Alternatively, the MSB kit 104B would be incubated in conditions known to the skilled artisan, preferably 18-48 hours. In a preferred embodiment, incubations of the same urine sample in differing media occur concurrently. For instance, the same blood sample 103A could be used to generate two blood preparations 103B, one in sterile water combined with media from the Aquagenx® CBT EC+TC P/A kit and one comprising MSB from an MSB kit. The 20-48-hour incubation specified by the Aquagenx® kit could then take place during the first 20-48 hours of the incubation necessary for culture in MSB to enable simultaneous readings.

Both incubation steps proceed to analytical step 440, wherein the urine preparation 102B and blood preparation 103B have become urine cultures 105 and blood cultures 106, respectively. The cultures are removed from the culturing environment so the user can analyze them according to the properties specific to the BMT kit 104. The method ends with terminal step 445.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and method stages which have been described and illustrated in order to explain the nature of this inventive subject matter can be made without departing from the principles and scope of the inventive subject matter.

What we claim is:

1. A method for detecting urinary tract infections, comprising steps of:
collecting a biological sample from a subject,
wherein said biological sample comprises a urine sample, and
wherein said subject is a human;
preparing one or more dilute preparations of said biological sample,
wherein at least one of said one or more dilute preparations comprises said biological sample diluted in sterile water,
adding solid media from a biological media test (BMT) kit to said one or more dilute preparations,
wherein said BMT kit is a drinking water test kit;
incubating said one or more dilute preparations with said solid media;
analyzing said one or more dilute preparations for indicia of bacterial presence to generate an assessment,
wherein said one or more dilute preparations are compared to a diagnostic key for said BMT kit,
wherein said diagnostic key for said BMT kit comprises a turbidity key that indicates a presence of bacteria, a color key that indicates a presence of coliform species, and a fluorescence key that indicates a presence of E. coli; and
generating a diagnosis for said subject based on said assessment.

2. The method of claim 1, wherein said BMT kit is an Aquagenx® CBT EC+TC P/A Kit.

3. The method of claim 2, wherein said one or more dilute preparations with said solid media are incubated at an ambient temperature between 23° and 27° Celsius.

4. The method of claim 2, wherein said one or more dilute preparations with said solid media are incubated for a period of time lasting between 20 and 48 hours.

5. The method of claim 2, wherein said one or more dilute preparations comprises a dilute preparation comprising 10 μL urine and 100 mL sterile water.

6. The method of claim 1, wherein said one or more dilute preparations comprises an inoculated mannitol salt broth (MSB).

7. The method of claim 6, further comprising the steps of:
generating said inoculated MSB;
incubating said inoculated MSB;
analyzing said inoculated MSB for indicia of bacterial presence to generate an assessment,
wherein said inoculated MSB is compared to a diagnostic key for inoculated MSB,
wherein said diagnostic key for inoculated MSB comprises a turbidity key that indicates a presence of Staphylococcus species and a color key that indicates a presence of Staphylococcus aureus; and
generating a diagnosis for said subject based on said assessment.

8. The method of claim 7, wherein said MSB inoculated with said one or more preparations is incubated at 37° Celsius for a period lasting between 18 and 48 hours.

9. The method of claim 6, wherein said inoculated MSB is modified to comprise 10 g/L tryptone peptone and 1 g/L yeast extract, wherein said inoculated MSB is modified to comprise neither 10 g/L proteose peptone nor 1 g/L beef extract.

10. The method of claim 1, wherein said biological sample further comprises a blood sample.

11. The method of claim 10, wherein said one or more dilute preparations further comprises a dilute preparation comprising 5 mL blood and 100 mL sterile water.

12. A method for detecting urinary tract infections, comprising steps of:
collecting a biological sample from a subject,
wherein said biological sample comprises a urine sample, and
wherein said subject is a human;
preparing one or more dilute preparations of said biological sample,
wherein at least one of said one or more dilute preparations comprises said biological sample diluted in sterile water,
adding solid media from a biological media test (BMT) kit to said one or more dilute preparations comprising said biological sample in sterile water,
wherein said BMT kit is an Aquagenx® CBT EC+TC MPN Kit;
incubating said one or more dilute preparations with said solid media;
analyzing said dilute preparations for indicia of bacterial presence to generate an assessment,
wherein said one or more dilute preparations are compared to a diagnostic key for said BMT kit,
wherein said diagnostic key for said BMT kit comprises a turbidity key that indicates a presence of bacteria, a color key that indicates a presence of coliform species, and a fluorescence key that indicates a presence of E. coli; and
generating a diagnosis for said subject based on said assessment.

13. The method of claim 12, wherein said one or more dilute preparations comprises an inoculated mannitol salt broth (MSB).

14. The method of claim 13, further comprising the steps of:
generating said inoculated MSB;
incubating said inoculated MSB;
analyzing said inoculated MSB for indicia of bacterial presence to generate an assessment,
wherein said inoculated MSB is compared to a diagnostic key for inoculated MSB,
wherein said diagnostic key for inoculated MSB comprises a turbidity key that indicates a presence of Staphylococcus species and a color key that indicates a presence of Staphylococcus aureus; and
generating a diagnosis for said subject based on said assessment.

15. The method of claim 14, wherein said inoculated MSB is incubated at 37° Celsius for a period lasting between 18 and 48 hours.

16. The method of claim 13, wherein said inoculated MSB is modified to comprise 10 g/L tryptone peptone and 1 g/L yeast extract, wherein said inoculated MSB comprises neither 10 g/L proteose peptone nor 1 g/L beef extract.

17. The method of claim 12, wherein said biological sample further comprises a blood sample.

18. A method for detecting bacteremia, comprising:
collecting a biological sample from a subject,
wherein said biological sample comprises a blood sample,
wherein said subject is a human;
generating an inoculated mannitol salt broth (MSB) comprising said biological sample,
wherein said inoculated MSB comprises 10 g/L tryptone peptone and 1 g/L yeast extract,
wherein said inoculated MSB comprises neither 10 g/L proteose peptone nor 1 g/L beef extract;
incubating said inoculated MSB;
analyzing said inoculated MSB for indicia of bacterial presence to generate an assessment,
wherein said inoculated MSB is compared to a diagnostic key for inoculated MSB,
wherein said diagnostic key for inoculated MSB comprises a turbidity key that indicates a presence of Staphylococcus species and a color key that indicates a presence of Staphylococcus aureus; and
generating a diagnosis for said subject based on said assessment.

19. The method of claim 18, further comprising:
preparing one or more dilute preparations of said biological sample,
wherein at least one of said one or more dilute preparations comprises said biological sample diluted in sterile water,
adding solid media from a biological media test (BMT) kit to said one or more dilute preparations,
wherein said BMT kit is an Aquagenx® CBT EC+TC P/A Kit;
incubating said one or more dilute preparations with said solid media;
analyzing said one or more dilute preparations for indicia of bacterial presence to generate an assessment,
wherein said one or more dilute preparations are compared to a diagnostic key for said BMT kit,
wherein said diagnostic key for said BMT kit comprises a turbidity key that indicates a presence of bacteria, a color key that indicates a presence of coliform species, and a fluorescence key that indicates a presence of E. coli; and
generating a diagnosis for said subject based on said assessment.

20. The method of claim 18, further comprising:
preparing one or more dilute preparations of said biological sample,
wherein at least one of said one or more dilute preparations comprises said biological sample diluted in sterile water,
adding solid media from a biological media test (BMT) kit to said one or more dilute preparations,
wherein said BMT kit is an Aquagenx® CBT EC+TC MPN Kit;
incubating said one or more dilute preparations with said solid media;
analyzing said one or more dilute preparations for indicia of bacterial presence to generate an assessment,
wherein said one or more dilute preparations are compared to a diagnostic key for said BMT kit,
wherein said diagnostic key for said BMT kit comprises a turbidity key that indicates a presence of bacteria, a color key that indicates a presence of coliform species, and a fluorescence key that indicates a presence of E. coli; and
generating a diagnosis for said subject based on said assessment.
